(19) United States
(12) Patent Application Publication
(10) Pub. No.: US 2026/0264818 A1
(43) Pub. Date: Sep. 10, 2026

(54) APPARATUS AND METHOD FOR MONITORING CHARACTERISTICS OF A LIQUID BODY

(71) Applicant: GeoKimika Energy Services, LLC, Conroe, TX (US)

(72) Inventors: Tanhee Galindo, Conroe, TX (US); Kristin Hatch, Midland, TX (US); Zachary Phillip Paul Smith, Hot Springs National Park, AR (US)

(21) Appl. No.: 19/555,551

(22) Filed: Mar. 3, 2026

Related U.S. Application Data
(60) Provisional application No. 63/767,838, filed on Mar. 6, 2025.

(51) Int. Cl. B63B 79/15; B63B 25/08; B63H 21/17; B63H 25/06; G01C 21/20; G01N 1/14; G01N 33/18

(52) U.S. Cl. CPC B63B 79/15; B63B 25/08; B63H 21/17; B63H 25/06; G01C 21/203; G01N 1/14; G01N 33/18

(57) ABSTRACT

An apparatus for monitoring characteristics of a liquid body includes a vessel with a propulsion system, a position-detection device configured to determine a location of the vessel, at least one sensor for capturing data about the liquid body, and a processing element. The processing element is configured to divide a map of the liquid body into a grid with sections, cause the propulsion system to propel the vessel along a path that intersects each of the sections, and direct a communication element to transmit the captured data in association with corresponding position data.

### CROSS-REFERENCE TO RELATED APPLICATIONS

The current patent application is a non-provisional utility patent application which claims priority benefit of earlier-filed U.S. Provisional Application Ser. No. 63/767,838; titled “APPARATUS AND METHOD FOR MONITORING CHARACTERISTICS OF A LIQUID BODY”; and filed Mar. 6, 2025. The Provisional Application is hereby incorporated by reference, in its entirety, into the current patent application.

### BACKGROUND OF THE INVENTION

Liquid bodies, such as holding ponds, retention basins, lagoons, settling ponds, or the like are often used in oil field operations and/or in processing plant operations. Such liquid bodies are vast water systems that expand across large geographical areas. In order to avoid overly stagnant conditions, environmental contamination, or conditions that otherwise impede use of the liquid in operations, operators must monitor various characteristics of the liquid. When certain conditions are detected in the water, operators must add counteracting or remedial chemicals to the water. However, it is difficult to distribute the additives in a uniform manner due to the large surface area of the liquid bodies. For example, some holding ponds are over 150 meters by 150 meters and have depths exceeding 10 meters. Additionally, due to the nature of such retained bodies of water, there is limited water flow for adequate mixing and dissolving of the added chemicals.

Thus, there is a need for improved techniques for maintaining acceptable conditions in such liquid bodies. This background discussion is intended to provide information related to the present invention which is not necessarily prior art.

### SUMMARY OF THE INVENTION

Embodiments of the current invention address one or more of the above-mentioned problems and provide a distinct advance in the art of monitoring characteristics of a liquid body.

One embodiment of the invention is an apparatus for monitoring characteristics of a liquid body. The apparatus includes a vessel, a propulsion system, at least one sensor, a communication element, a position-detection device, and a processing element. The vessel defines a hull. The propulsion system is secured to the vessel and is operable to propel the vessel in the liquid body. The at least one sensor is supported by the vessel and is configured to capture data associated with the liquid body. The position-detection device is configured to generate position data of the vessel in the liquid body. The communication element is configured to transmit one or more signals representative of the captured data and/or the position data. The processing element is in communication with the communication element and is configured to divide a map of the liquid body into a grid with sections, direct the propulsion system and the actuator to cause the vessel to traverse a path that intersects each of the sections, and direct the communication element to transmit the captured data in association with corresponding position data. By gathering data at different locations in the liquid body, operators can supplement the liquid body with appropriate chemicals only in areas where it is necessary based on the captured data.

Another embodiment of the invention is a method of monitoring characteristics of a liquid body. The method includes capturing data associated with the liquid body using the apparatus described above. The method further includes receiving, via a remote device, the captured data in association with the corresponding position data; and displaying, via a user interface of the remote device, the captured data in association with the corresponding position data.

Another embodiment of the invention is a system for monitoring characteristics of a liquid body. The system includes the apparatus described above, and further comprises a remote device in wireless communication with the apparatus. The remote device includes a communication element, a memory element, a user interface, and a processing element. The communication element is in wireless communication with the apparatus and is configured to receive a signal representative of the captured data. The memory element is configured to store the captured data, and the user interface is configured to display the captured data. The processing element is configured to render a graph of the captured data in association with the corresponding position data on the user interface.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the current invention will be apparent from the following detailed description of the embodiments and the accompanying drawing figures.

### BRIEF DESCRIPTION OF DRAWINGS

Embodiments of the current invention are described in detail below with reference to the attached drawing figures, wherein:

FIG. 1 is a perspective view of an apparatus constructed according to an embodiment of the present invention;

FIG. 2 is an elevated perspective view of a top side of the apparatus of FIG. 1 with a cover removed from a hull of the apparatus;

FIG. 3 is a side elevation view of the apparatus of FIG. 1 positioned in a liquid body;

FIG. 4 is a block diagram depicting selected components of the apparatus of FIG. 1;

FIG. 5 is a perspective view of an apparatus constructed according to another embodiment of the present invention;

FIG. 6 is a perspective view of the apparatus of FIG. 5 with solar panels removed;

FIG. 7 is schematic diagram of a user interface of a remote device in which embodiments of the present invention may be implemented;

FIG. 8 is a schematic diagram of the user interface of FIG. 7 with a heat map rendered thereon; and

FIG. 9 is a flowchart depicting exemplary steps of a method according to an embodiment of the present invention.

The drawing figures do not limit the current invention to the specific embodiments disclosed and described herein. The drawings are not necessarily to scale, emphasis instead being placed upon clearly illustrating the principles of the invention.

### DETAILED DESCRIPTION OF THE INVENTION

The following detailed description of the technology references the accompanying drawings that illustrate specific embodiments in which the technology can be practiced. The embodiments are intended to describe aspects of the technology in sufficient detail to enable those skilled in the art to practice the technology. Other embodiments can be utilized and changes can be made without departing from the scope of the current invention. The following detailed description is, therefore, not to be taken in a limiting sense. The scope of the current invention is defined only by the appended claims, along with the full scope of equivalents to which such claims are entitled.

Due to the large surface area of retained liquid bodies, the distribution of particles, dissolved oxygen, etc. is not always uniform. Additionally, it is impractical, inefficient, and in some cases, hazardous to repeatedly traverse the liquid bodies to obtain higher granularity in the data. Thus, embodiments of the invention allow for capturing data at different locations in a liquid body to generate a heat map of relevant metrics so that operators can precisely supplement the liquid body where needed to remedy unfavorable conditions in the liquid body. The liquid may comprise an aqueous (water-based) fluid having components dissolved, suspended, or otherwise present therein other than water, as discussed in U.S. Patent Application Publication No. 2024/0218786, which is incorporated by reference herein in its entirety. For example, the liquid may comprise an aqueous (water-based) fluid comprising water as the majority component (i.e., at least 50%, at least 60%, at least 70%, at least 80%, at least 90%, at least 95%, at least 97%, at least 98%, or at least 99% by weight), which may further comprise sand, alcohols (e.g., methanol, ethylene glycol), thickening agents, acids (e.g., hydrochloric acid, acetic acid), salts (e.g., sodium chloride), friction reducers (e.g., polyacrylamide), anti-corrosion chemicals, anti-microbials, foaming agents, scale inhibitors, and/or pH buffers (e.g., KOH, etc.).

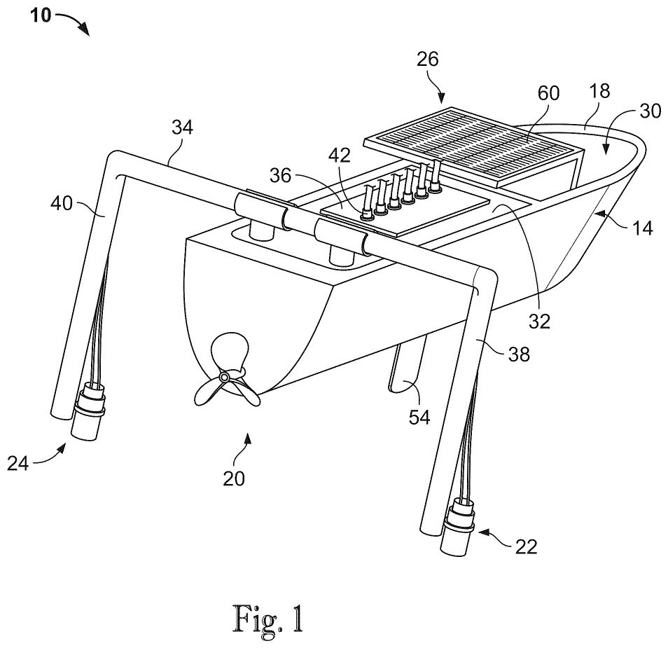

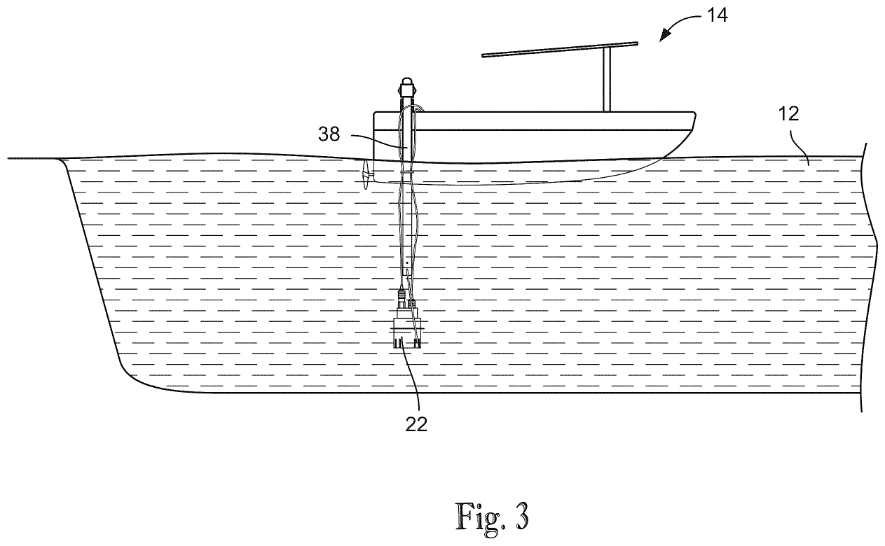

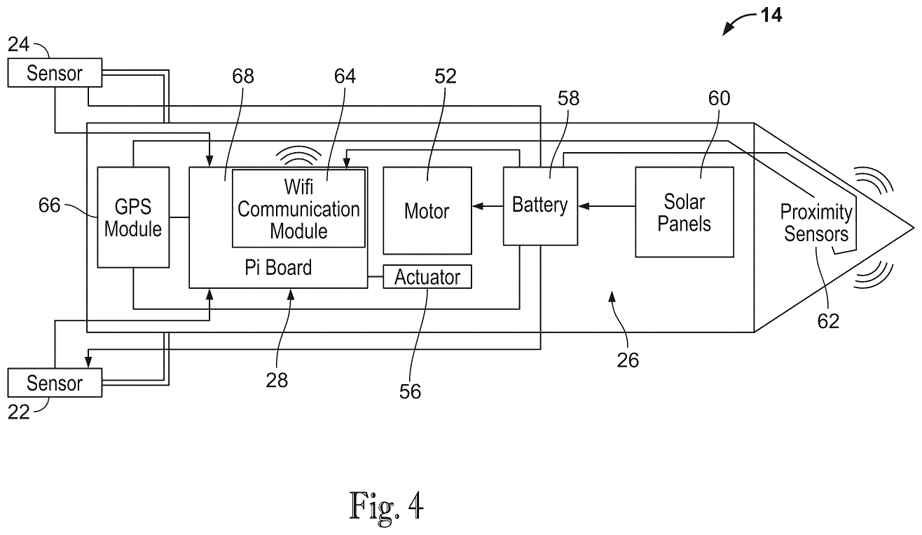

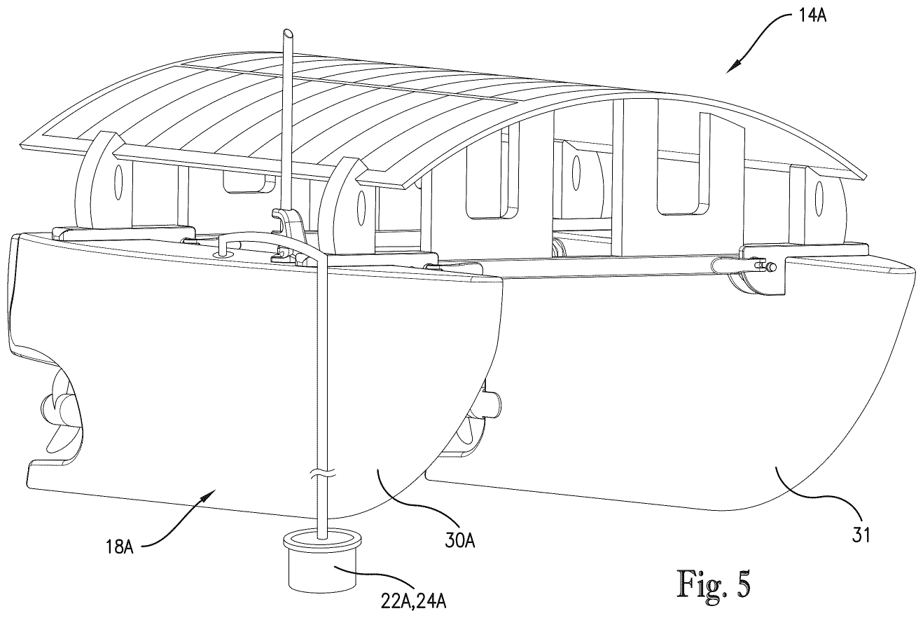

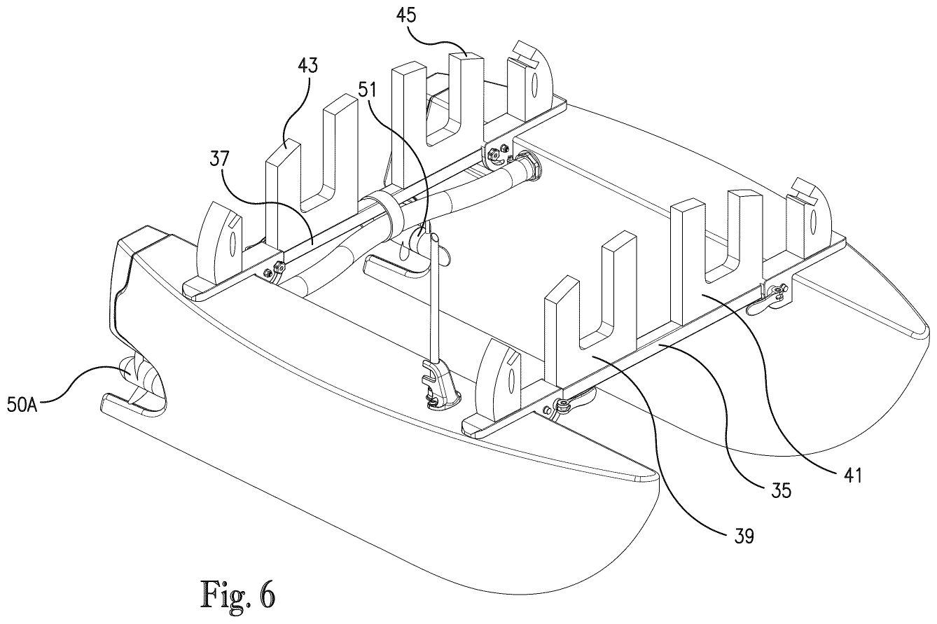

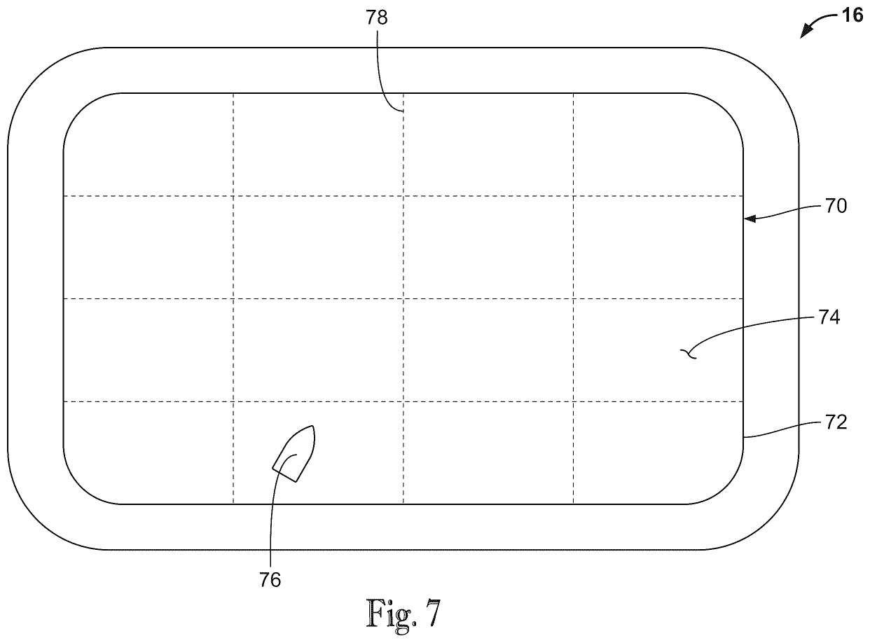

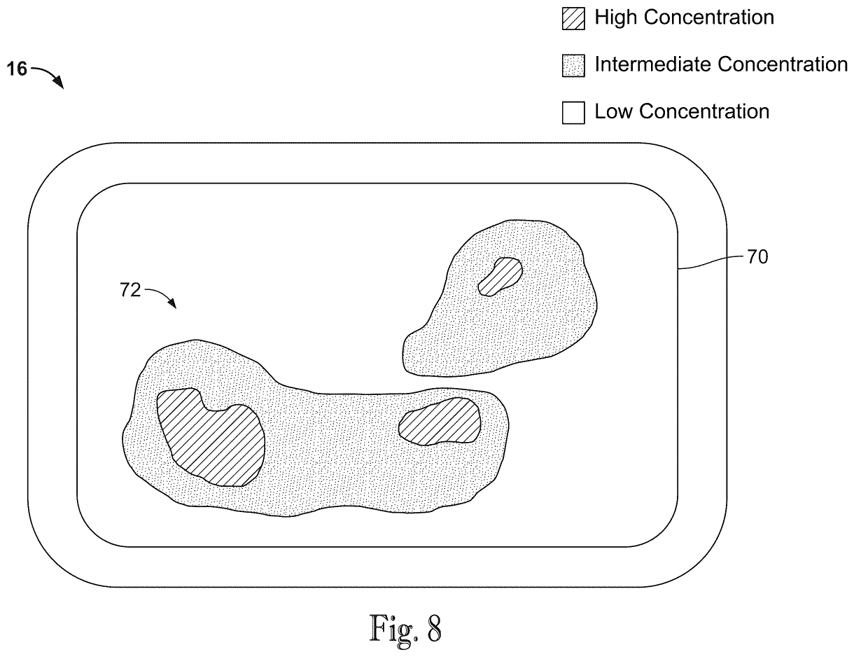

Turning to FIG. 1, a system 10 constructed according to an embodiment of the present invention is configured to monitor characteristics of a liquid body 12 (depicted in FIG. 3). The liquid body 12 may be a natural body of water (such as a lake, river, or sea) or a man-made liquid body, such as a holding pond used in an oil field operation, a retention basin, a lagoon, a settling pond, or the like. The liquid body 12 may be any size and have a variable or uniform depth. The characteristics may include an acidity (pH), oxidation-reduction potential (ORP), temperature, conductivity, total dissolved solids, scaling, corrosion, turbidity, dissolved oxygen, dissolved carbon dioxide, analyte concentrations, pressure, flow rate, specific gravity, oxidizer concentration (e.g., chlorine, chlorine dioxide, peracetic acid, etc.), and/or others. Sensor readings for such characteristics can provide on-site and off-site operators with valuable information regarding fluid quality, fluid conditions, sediment (e.g., sand) levels, and/or chemical levels of various fluids related to hydraulic fracturing operations. In one or more embodiments, the system 10 includes an apparatus 14 and one or more remote devices 16 (depicted in FIGS. 7 and 8). The apparatus 14 broadly comprises a vessel 18, which may be a single or multi-hulled watercraft (as depicted in FIGS. 5 and 6) or other vehicle such as a hovercraft, a propulsion and steering system 20, one or more sensor probes 22, 24, a power system 26, and a control system 28 (depicted in FIG. 4).

In one or more embodiments, the vessel 18 is a boat operable to float on the liquid body and support the various components of the apparatus 14. The vessel 18 may define one or more hulls 30 and includes a top deck 32, a boom 34 supported on the top deck 32, and a cover 36 for closing an opening of one or more of the hulls 30. The boom 34 extends laterally across the top deck 32 and includes two downwardly extending arms 38, 40 operable to extend into the liquid body and support the sensor probes 22, 24 submerged in the liquid body. The cover 36 has one or more ports 42 for providing an electrical connection between the propulsion and steering system 20, the sensor probes 22, 24, the power system 26, and/or the control system 28.

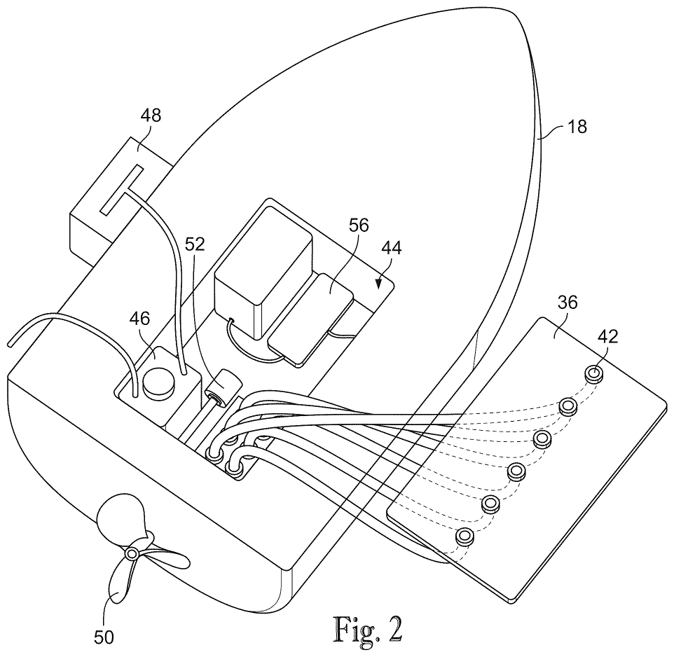

Turning to FIG. 2, the cover 36 is removably secured over a cavity 44 of the hull 30 for providing access to components stored therein. In one or more embodiments, the vessel 18 includes a storage container 46 and a pump 48. The storage container 46 is for storing samples of the liquid body, and the pump 48 is configured to direct liquid from the liquid body into the storage container 46. In one or more embodiments, the pump 48 is a peristaltic pump. This allows operators to remotely direct the apparatus 14 to draw in a sample of the liquid body in the container 46, and retrieve the sample from the apparatus 14 to perform an on-site and/or lab test to verify readings from the sensor probes 22, 24.

The propulsion and steering system 20 is configured to move the vessel 18 in the liquid body. In one or more embodiments, the propulsion and steering system 20 comprises a propeller 50 secured to the vessel 18, a motor 52 for actuating the propeller 50 to generate a propulsive force, a rudder 54 (depicted in FIG. 1) operable to help steer the vessel 18, and an actuator 56 operable to adjust the rudder 54. While the propeller 50 is depicted as an outboard propeller with external fins, the propulsion and steering system 20 may use any technology known in the art, including an inboard system, a stern drive, a jet drive, or the like. Additionally, the rudder 54 may alternatively be combined with the propeller so that the actuator adjusts a yaw of the propeller thereby steering vessel. The steering system may also comprise thrusters or jets that can be individually actuated to steer the vessel.

Turning briefly back to FIG. 1, the sensor probes 22, 24 are supported by the vessel 18 and configured to capture data associated with the liquid body. In one or more embodiments, the sensor probes 22, 24 hang from the ends of the arms 38, 40 of the boom 34. In one or more embodiments, each of the sensor probes 22, 24 includes an array of sensors comprising five or more sensors for capturing data about the liquid body. As discussed above, the sensors may include any number of sensors known in the art for detecting characteristics of the liquid body, such as an acidity or pH of the liquid, a salinity of the liquid, a depth of the liquid body, a level of dissolved oxygen, a concentration of a particle substances, a temperature of the liquid, or the like. Turning to FIG. 3, the sensor probes 22, 24 may be operable to be lowered to a number of depths in the liquid body. For example, the vessel 18 may include reels or winches for lowering the sensor probes 22, 24. In one or more embodiments, the sensor probes 22, 24 may be operable to be lowered to a depth of around 61 centimeters. In one or more embodiments, the sensor probes 22, 24 may be operable to be lowered to a depth of at least 3 meters.

Turning to FIG. 4, the power system 26 is operable to provide power to various components of the apparatus 14, such as the propulsion and steering system 20, the sensor probes 22, 24, and the control system 28. In one or more embodiments, the power system 26 includes one or more energy storage devices 58 and one or more solar panels 60 for charging the energy storage device 58. The energy storage device 58 may comprise a battery, such as a LiFePO4 battery, a capacitor, or the like. The solar panels 60 enable the apparatus 14 to remain in operation for significantly longer periods of time.

The control system 28 is configured to control operations of the apparatus 14. The control system 28 may comprise one or more proximity sensors 62, one or more communication elements 64, one or more memory elements, one or more position-detection devices 66, and one or more processing elements 68. The proximity sensors 62 are secured to the vessel 18 and are in communication with the processing element 68. The proximity sensor 62 are operable to help prevent the apparatus 14 from colliding with obstacles. The communication element is 64 configured to transmit one or more signals representative of the data captured by the sensor probes 22, 24. The memory element is in communication with the processing element 68 and is configured to store the captured data in association with corresponding position data from the position-detection device 66. The position-detection device 66 is configured to generate position data of the vessel 18 in the liquid body. In one or more embodiments, the position-detection device 66 is a global positioning system (GPS) configured to determine GPS coordinates associated with a current position of the vessel 18.

The processing element 68 is in communication with the communication element 64 and is configured to divide a map of the liquid body into a grid with sections. The map may be stored on the memory element. In one or more embodiments, the processing element 68 is configured to divide a top surface of the map of the liquid body into the grid so that each of the sections represents a desired area of the top surface of the liquid body. In one or more embodiments, the map is a perimeter of the liquid body. In some embodiments, the processing element 68 is configured to determine the perimeter of the liquid body automatically using the proximity sensors 62 and by directing the propulsion and steering system 20 to travel along the boundary of the liquid body. Once the map of the liquid body is determined, the processing element 68 may be configured to divide the map with grid lines to form sections according to a predetermined size or grid unit. The processing element 68 is configured to determine a path to obtain readings from a plurality of grid sections. In one or more embodiments, the processing element 68 is configured to determine a path that intersects each of section of the grid.

The processing element 68 is configured to direct the propulsion and steering system 20 to cause the vessel to traverse the path. In one or more embodiments, the processing element 68 is configured to use the sensor probes 22, 24 to capture data in the grids along the path. The processing element 68 may be configured to capture data while the vessel 18 is in motion. Alternatively or additionally, the processing element 68 is configured to direct the propulsion and steering system 20 to make periodic stops to capture data via the sensor probes 22, 24. The processing element 68 may be configured to direct the propulsion and steering system 20 to position the probes 22, 24 within a tolerance of a center point of the grids to capture the data. In one or more embodiments, the tolerance is about three meters, about two meters, or about one meter. While traversing the liquid body, the processing element 68 is configured to receive an indication of a distance to obstacles based on signals from the proximity sensors 62. The processing element 68 is configured to direct the propulsion and steering system 20 to cause the vessel 18 to maintain a predetermined distance from obstacles.

In one or more embodiments, the processing element 68 is configured to perform one or more operations to enhance the data. For example, the processing element 68 may be configured to cause the apparatus 14 to repeat the path one or more times. In one or more embodiments, the processing element 68 is configured to cause the sensor probes 22, 24 to capture at least two data types in each grid. In one or more embodiments, the processing element 68 is configured to direct the pump 48 to collect a sample into the storage container 46, receive the location data where the sample was taken from the position-detection device 66, and store the location data of the vessel 18 where the sample was taken and a time during which the sample was taken.

In one or more embodiments, the processing element 68 is configured to process and/or relay at least some of the data. For example, the processing element 68 may generate a heat map of the map of the liquid body according to the captured data and the corresponding position data. The processing element 68 may be configured to direct the communication element 64 to transmit the captured data in association with corresponding position data and/or temporal data. The processing element 68 may also be configured to direct the communication element 64 to transmit the heat map.

In one or more embodiments, the processing element 68 is configured to direct the propulsion and steering system 20 to cause the vessel to travel to a designated location in the liquid body. The designated location may be at a location along an edge of the liquid body. This allows operators to be able to collect a sample, perform maintenance on the apparatus 14, or otherwise collect the apparatus 14.

An apparatus 14A constructed in accordance with another embodiment of the invention is shown in FIGS. 5 and 6. The apparatus 14A may comprise substantially similar components as apparatus 14; thus, the components of apparatus 14A that correspond to similar components in apparatus 14 have an ‘A’ appended to their reference numerals.

Turning to FIG. 5, the apparatus 14A includes all the features of apparatus 14 except that the vessel 18A is multi-hulled to improve stability and includes two or more hulls 30A, 31. The solar panels 60A extend between the hulls 30A, 31 to cover the components underneath and supply power to the power system of the apparatus 14A. The sensor probes 22A, 24A extend over the side of one or more of the hulls 30A, 31 and are suspended in the liquid body below. Turning to FIG. 6, the hulls 30A, 31 are connected via one or more frame structures 35, 37 extending between the hulls 30A, 31. The apparatus 14A includes one or more mounting brackets 39, 41, 43, 45 attached to the frame structures 35, 37 for supporting the solar panels 60A. The propulsion system includes a propellor 50A, 51 and corresponding motor operatively associated with their respective hulls 30A, 31. The control system may be configured to selectively activate the motors of the propellors 50A, 51 to steer the apparatus 14A. This obviates the need for a rudder.

Turning to FIG. 7, the remote device 16 is in wireless communication with the apparatus 14, 14A. The remote device 16 may be any computing device known in the art, such as a mobile phone, tablet, laptop computer, desktop computer, server, or the like. The remote device 16 comprises a communication element, a memory element, a user interface 70, and a processing element. The communication element is configured to receive one or more signals representative of the captured data, location data, and/or corresponding temporal data. The memory element of the remote device 16 is configured to store the received data. The user interface 70 is configured to display the captured data and location data. The processing element of the remote device 16 is configured to render a graph or map 72 of the liquid body 74 on the user interface 70. The map 72 of the liquid body may include an icon 76 representative of a location of the apparatus in the liquid body and the grid lines 78.

Turning to FIG. 8, the remote device 16 is configured to render a graph or map 72 of the captured data in association with the corresponding position data on the user interface 70. The map 72 may be a heat map of the liquid body according to the captured data and the corresponding position data. For example, the map 72 may display shapes representative of regions of the liquid body having certain characteristics, such as high, intermediate, or low concentrations of a detected compound. The heat map may be color coordinated including displaying red, orange, or green regions depending on the data captured in the area. For example, green may represent areas with acceptable conditions, orange may represent unacceptable conditions, and red may represent dangerous conditions. The remote device 16 may be configured to additionally receive the raw data captured by the probes. The apparatus 14, 14A may be configured to provide periodic reports of the data captured, such as on a daily basis, to the remote device 16. The apparatus 14, 14A may also be configured to generate alerts when measurements exceed certain thresholds and transmit the alerts to the remote device 16.

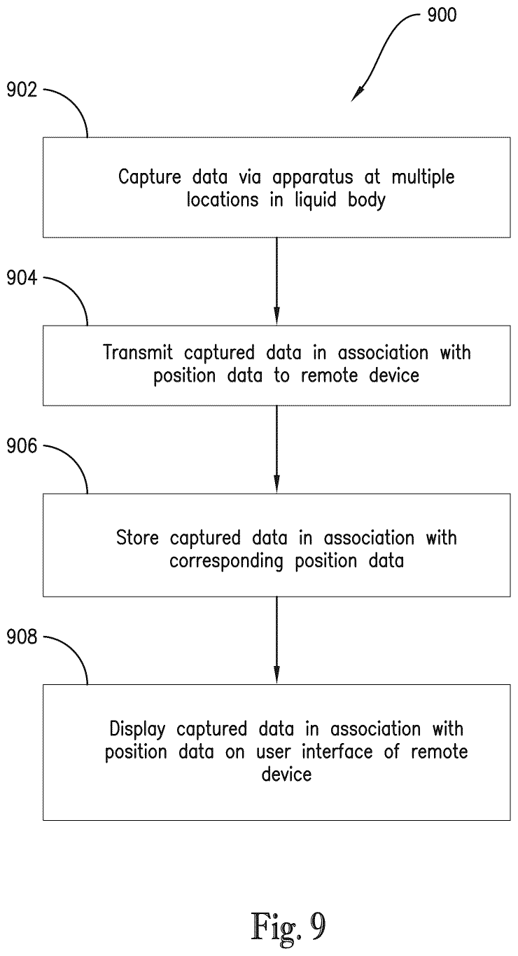

The flow chart of FIG. 9 depicts the steps of an exemplary method 900 of monitoring characteristics of a liquid body. In some alternative implementations, the functions noted in the various blocks may occur out of the order depicted in FIG. 9. For example, two blocks shown in succession in FIG. 9 may in fact be executed substantially concurrently, or the blocks may sometimes be executed in the reverse order depending upon the functionality involved. In addition, some steps may be optional.

The method 900 is described below, for ease of reference, as being executed by exemplary devices and components introduced with the embodiments illustrated in FIGS. 1-8. The steps of the method 900 may be performed by the control system through the utilization of processors, transceivers, hardware, software, firmware, or combinations thereof. However, some of such actions may be distributed differently among such devices or other devices without departing from the spirit of the present invention. Control of the system may also be partially implemented with computer programs stored on one or more non-transient computer-readable medium(s). The computer-readable medium(s) may include one or more executable programs stored thereon, wherein the program(s) instruct one or more processing elements to perform all or certain of the steps outlined herein. The program(s) stored on the computer-readable medium(s) may instruct processing element(s) to perform additional, fewer, or alternative actions, including those discussed elsewhere herein.

Referring to step 902, data associated with the liquid body is captured via an apparatus constructed according to embodiments described herein. This step may include building a grid of the liquid body, via the apparatus. For example, the coordinates of the perimeter of the liquid body may be known, and the processing element of the apparatus may overlay a grid on a map of the perimeter. This step may include automatically moving the apparatus across the liquid body along a path that intersects various points on the grid for data collection at each point. The captured data, the position data, and the temporal data may be stored on the memory element of the apparatus. This step may also include taking a sample of the liquid via the pump and storing the sample in the container. This step may include recording a location and time at which the sample was taken.

Referring to step 904, the captured data, the corresponding position data, and the temporal data are received at the remote device. The remote device may receive the data via wireless communication with the apparatus.

Referring to step 906, the captured data is stored, via the processing element of the remote device, in association with the corresponding position data and/or temporal data. This step may also include relaying data to another device.

Referring to step 908, the captured data is displayed, via a user interface of the remote device, in association with the corresponding position data. This step may include generating the heat map via the processing element of the apparatus and/or via the processing element of the remote device.

The method 900 may include additional, less, or alternate steps and/or device(s), including those discussed elsewhere herein. For example, step 902 may be repeated a periodically. Additionally, the method 900 may include a step of receiving one or more instructions from the user interface of the remote device to relay to the apparatus, including instructions for the apparatus to travel to a predetermined location and/or take a sample of the liquid body.

Embodiments of the method 900 may further comprise one or more intervention steps, which may be used to change one or more characteristics of the liquid body in response to the captured data generated during the monitoring steps described above. Exemplary intervention steps may include adjusting the flow rate of the fluid flowing into the liquid body and/or adjusting the concentration of one or more additives (such as those described herein) in the liquid body. In certain such embodiments, the methods further comprise adjusting the amount of one or more additives introduced into the liquid body generally and/or at targeted regions in the liquid body at which unacceptable conditions were detected. The adjusting step may comprise introducing a greater amount of a certain additive to the liquid body at those locations, for example to increase the concentration of that additive in that location in the liquid body, or the adjusting step may comprise introducing a lesser amount of a certain additive to that location in the liquid body (or eliminating introduction of that additive altogether), for example to decrease the concentration of that additive in the location of the liquid body. The intervention step may include using the apparatus described herein and/or other remote-controlled vessels for delivering the treatment to the specific location in the liquid body having unacceptable conditions.

For example, if the heat map presents areas where the pH is outside of an acceptable range, operators can direct via wireless instructions and/or programming enabling automation that cause the same or a different vessel to spot treat in that area, as opposed to broadly dumping a treatment into the liquid body indiscriminately, which may induce unacceptable conditions at different locations in the liquid body.

Throughout this specification, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the current invention can include a variety of combinations and/or integrations of the embodiments described herein.

Although the present application sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as computer hardware that operates to perform certain operations as described herein.

In various embodiments, computer hardware, such as a processing element, may be implemented as special purpose or as general purpose. For example, the processing element may comprise dedicated circuitry or logic that is permanently configured, such as an application-specific integrated circuit (ASIC), or indefinitely configured, such as an FPGA, to perform certain operations. The processing element may also comprise programmable logic or circuitry (e.g., as encompassed within a general-purpose processor or other programmable processor) that is temporarily configured by software to perform certain operations. It will be appreciated that the decision to implement the processing element as special purpose, in dedicated and permanently configured circuitry, or as general purpose (e.g., configured by software) may be driven by cost and time considerations.

Accordingly, the term “processing element” or equivalents should be understood to encompass a tangible entity, be that an entity that is physically constructed, permanently configured (e.g., hardwired), or temporarily configured (e.g., programmed) to operate in a certain manner or to perform certain operations described herein. Considering embodiments in which the processing element is temporarily configured (e.g., programmed), each of the processing elements need not be configured or instantiated at any one instance in time. For example, where the processing element comprises a general-purpose processor configured using software, the general-purpose processor may be configured as respective different processing elements at different times. Software may accordingly configure the processing element to constitute a particular hardware configuration at one instance of time and to constitute a different hardware configuration at a different instance of time.

The processing element may include processors, microprocessors (single-core and multi-core), microcontrollers, DSPs, field-programmable gate arrays (FPGAs), analog and/or digital application-specific integrated circuits (ASICs), or the like, or combinations thereof. The processing element may generally execute, process, or run instructions, code, code segments, software, firmware, programs, applications, apps, processes, services, daemons, or the like. The processing element may also include hardware components such as finite-state machines, sequential and combinational logic, and other electronic circuits that can perform the functions necessary for the operation of the current invention. The processing element may be in communication with the other electronic components through serial or parallel links that include address busses, data busses, control lines, and the like.

Computer hardware components, such as communication elements, memory elements, processing elements, and the like, may provide information to, and receive information from, other computer hardware components. Accordingly, the described computer hardware components may be regarded as being communicatively coupled. Where multiple of such computer hardware components exist contemporaneously, communications may be achieved through signal transmission (e.g., over appropriate circuits and buses) that connect the computer hardware components. In embodiments in which multiple computer hardware components are configured or instantiated at different times, communications between such computer hardware components may be achieved, for example, through the storage and retrieval of information in memory structures to which the multiple computer hardware components have access. For example, one computer hardware component may perform an operation and store the output of that operation in a memory device to which it is communicatively coupled. A further computer hardware component may then, at a later time, access the memory device to retrieve and process the stored output. Computer hardware components may also initiate communications with input or output devices, and may operate on a resource (e.g., a collection of information).

The memory device or element may include data storage components, such as read-only memory (ROM), programmable ROM, erasable programmable ROM, random-access memory (RAM) such as static RAM (SRAM) or dynamic RAM (DRAM), cache memory, hard disks, floppy disks, optical disks, flash memory, thumb drives, universal serial bus (USB) drives, or the like, or combinations thereof. In some embodiments, the memory element may be embedded in, or packaged in the same package as, the processing element. The memory element may include, or may constitute, a “computer-readable medium”. The memory element may store the instructions, code, code segments, software, firmware, programs, applications, apps, services, daemons, or the like that are executed by the processing element.

The communication element may generally allow communication with systems and/or external devices. The communication element may include signal or data transmitting and receiving circuits, such as antennas, amplifiers, filters, mixers, oscillators, digital signal processors (DSPs), and the like. The communication element may establish communication wirelessly by utilizing RF signals and/or data that comply with communication standards such as cellular 2G, 3G, 4G, 5G, or LTE, WiFi, WiMAX, Bluetooth®, BLE, or combinations thereof. The communication element may be in communication with the processing element and the memory element.

The user interface generally allows the user to utilize inputs and outputs to interact with the device and is in communication with the one or more processing element. Inputs may include buttons, pushbuttons, knobs, jog dials, shuttle dials, directional pads, multidirectional buttons, switches, keypads, keyboards, mice, joysticks, microphones, or the like, or combinations thereof. The outputs of the present invention may include a display and/or any number of additional outputs, such as audio speakers, lights, dials, meters, printers, or the like, or combinations thereof, without departing from the scope of the present invention.

The various operations of example methods described herein may be performed, at least partially, by one or more processing elements that are temporarily configured (e.g., by software) or permanently configured to perform the relevant operations. Whether temporarily or permanently configured, such processing elements may constitute processing element-implemented modules that operate to perform one or more operations or functions. The modules referred to herein may, in some example embodiments, comprise processing element-implemented modules.

Similarly, the methods or routines described herein may be at least partially processing element-implemented. For example, at least some of the operations of a method may be performed by one or more processing elements or processing element-implemented hardware modules. The performance of certain of the operations may be distributed among the one or more processing elements, not only residing within a single machine, but deployed across a number of machines. In some example embodiments, the processing elements may be located in a single location (e.g., within a home environment, an office environment or as a server farm), while in other embodiments the processing elements may be distributed across a number of locations.

Unless specifically stated otherwise, discussions herein using words such as “processing,” “computing,” “calculating,” “determining,” “presenting,” “displaying,” or the like may refer to actions or processes of a machine (e.g., a computer with a processing element and other computer hardware components) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112(f) unless traditional means-plus-function language is expressly recited, such as “means for” or “step for” language being explicitly recited in the claim(s).

Although the technology has been described with reference to the embodiments illustrated in the attached drawing figures, it is noted that equivalents may be employed and substitutions made herein without departing from the scope of the technology as recited in the claims.

Having thus described various embodiments of the technology, what is claimed as new and desired to be protected by Letters Patent includes the following:

1. An apparatus for monitoring characteristics of a liquid body, the apparatus comprising:
a vessel defining one or more hulls;
a propulsion system secured to the vessel and operable to propel the vessel in the liquid body;
at least one sensor supported by the vessel and configured to capture data associated with the liquid body;
a position-detection device configured to generate position data of the vessel in the liquid body;
a communication element configured to transmit one or more signals representative of the
captured data and the position data; and
one or more processing elements in communication with the communication element and configured to:
divide a map of the liquid body into a grid with sections,
direct the propulsion system to cause the vessel to traverse a path that intersects each of the sections, and
direct the communication element to transmit the captured data in association with corresponding position data.

2. The apparatus of claim 1, further comprising a memory element in communication with the one or more processing elements, wherein the one or more processing elements is configured to store the captured data in association with corresponding position data on the memory element.

3. The apparatus of claim 1, wherein captured data is associated with at least one of a pH of the liquid body, a salinity of the liquid body, a depth of the liquid body, a level of dissolved oxygen, a concentration of particulate or dissolved substances, or a temperature of the liquid body.

4. The apparatus of claim 1, wherein the one or more hulls includes a first hull, a second hull, and a frame element extending between the first and second hulls, the propulsion system comprising:
a first propeller operatively associated with the first hull,
a first motor configured to drive the first propeller,
a second propeller operatively associated with the second hull, and
a second motor configured to drive the second propeller.

5. The apparatus claim 1, wherein the propulsion system comprises a rudder movably connected to the one or more hulls, an actuator configured to adjust an orientation of the rudder, a propeller operatively associated with the one or more hulls, and a motor configured to drive the propeller.

6. The apparatus of claim 1, wherein the one or more processing elements are configured to divide a top surface of the map of the liquid body into the grid so that each of the sections represent a desired area of the top surface of the liquid body.

7. The apparatus of claim 1, wherein the one or more processing elements are configured to generate a heat map of the liquid body according to the captured data and the corresponding position data.

8. The apparatus of claim 1, further comprising one or more proximity sensors secured to the vessel and in communication with the one or more processing elements, wherein the one or more processing elements are configured to receive an indication of a distance to an object based on one or more signals from the one or more proximity sensors and direct the propulsion system to cause the vessel to maintain a predetermined distance from the object.

9. The apparatus of claim 1, further comprising one or more energy storage devices located in the one or more hulls and operable to provide electrical power to the one or more processing elements, the propulsion system, the at least one sensor, the communication element, and the position-detection device.

10. The apparatus of claim 9, further comprising one or more solar panels supported on the vessel and operable to provide electrical energy to the one or more energy storage devices.

11. The apparatus of claim 1, further comprising:
a storage container located on the vessel; and
a pump configured to direct liquid from the liquid body into the storage container.

12. The apparatus of claim 1, wherein the one or more processing elements are configured to direct the propulsion system to cause the vessel to travel to a designated location in the liquid body.

13. A method of monitoring characteristics of a liquid body, the method comprising:
capturing, via an apparatus, data associated with the liquid body, the apparatus comprising:
a vessel defining one or more hulls;
a propulsion system secured to the vessel and operable to propel the vessel in the liquid body;
at least one sensor supported by the vessel and configured to capture data associated with the liquid body;
a position-detection device configured to generate position data of the vessel in the liquid body;
a communication element configured to transmit one or more signals representative of the captured data and the position data. and
one or more processing elements in communication with the communication element and configured to:
divide a map of the liquid body into a grid with sections,
direct the propulsion system to cause the vessel to traverse a path that intersects each of the sections, and
direct the communication element to transmit the captured data in association with corresponding position data;
receiving, via a remote device, the captured data in association with the corresponding position data; and
displaying, via a user interface of the remote device, the captured data in association with the corresponding position data.

14. The method of claim 13, further comprising periodically repeating the step of capturing, via the apparatus, the data associated with the liquid body according to a desired frequency.

15. The method of claim 14, further comprising storing, via the one or more processing element of the remote device, the captured data in association with the corresponding position data and corresponding temporal data.

16. The method of claim 13, further comprising displaying, via the user interface of the remote device, a heat map of the liquid body according to the captured data and the corresponding position data.

17. The method of claim 13, further comprising applying an additive to a portion of the liquid body associated with the captured data and the corresponding position data in which a detected condition of the portion of the liquid body is outside of an acceptable range.

18. A system for monitoring characteristics of a liquid body, the system comprising:
an apparatus comprising:
a vessel defining one or more hulls;
a propulsion system secured to the vessel and operable to propel the vessel in the liquid body;
at least one sensor supported by the vessel and configured to capture data associated with the liquid body;
a position-detection device configured to generate position data of the vessel in the liquid body;
a communication element configured to transmit one or more signals representative of the captured data and the position data; and
one or more processing elements in communication with the communication element and configured to:
divide a map of the liquid body into a grid with sections,
direct the propulsion system to cause the vessel to traverse a path that intersects each of the sections, and
direct the communication element to transmit the captured data in association with corresponding position data;
a remote device in wireless communication with the apparatus, the remote device comprising:
a communication element in wireless communication with the apparatus and configured to receive one or more signals representative of the captured data,
one or more memory elements configured to store the captured data,
a user interface configured to display the captured data, and
one or more processing elements configured to render a graph of the captured data in association with the corresponding position data on the user interface.

19. The system of claim 18, wherein the one or more processing elements of the apparatus are configured to generate a heat map of the liquid body according to the captured data and the corresponding position data and direct the communication element of the apparatus to transmit the heat map to the remote device, wherein the one or more processing elements of the remote device is configured to display the heat map on the user interface.

20. The system of claim 18, wherein the one or more processing elements of the remote device is configured to generate a heat map of the liquid body according to the captured data and the corresponding position data and display the heat map on the user interface.
